(19) United States
(12) Patent Application Publication
(10) Pub. No.: US 2015/0370996 A1
(43) Pub. Date: Dec. 24, 2015

(54) System for determining the need for Angiography in patients with symptoms of Coronary Artery disease

(71) Applicants: Roohallah Alizadehsani, Tabas (IR); Mohammad Javad Hosseini, Shiraz (IR); Zahra Alizadehsani, Tehran (IR); Mohammad Hassan Mohammadi, Shiraz (IR); Ozra Barati, Tabas (IR); Fahimeh Khozeimeh, Mashhad (IR)

(72) Inventors: Roohallah Alizadehsani, Tabas (IR); Mohammad Javad Hosseini, Shiraz (IR); Zahra Alizadehsani, Tehran (IR); Mohammad Hassan Mohammadi, Shiraz (IR); Ozra Barati, Tabas (IR); Fahimeh Khozeimeh, Mashhad (IR)

(21) Appl. No.: 14/312,648

(22) Filed: Jun. 23, 2014

(51) Int. Cl. G06F 19/00; A61B 6/00

(52) U.S. Cl. CPC G06F 19/3443; A61B 6/504

(57) ABSTRACT

The present invention relates to a system for determining the need for angiography in patients with symptoms of Coronary Artery Disease (CAD), and comprises a data mining algorithm that processes a dataset with a set of predetermined features, preferably 50 features. The system comprises a pre-processing phase and a main phase.

### BACKGROUND OF THE INVENTION

Cardiovascular diseases are extremely widespread and account for 17 million deaths in the world per annum. Coronary Artery Disease (CAD) is one of such diseases with an annual mortality rate of about 7 million. Thus, early diagnosis of CAD is of global vital importance. A patient has CAD, when at least one of the arteries, Left Anterior Descending (LAD), Left Circumflex (LCX), or Right Coronary Artery (RCA), is blocked. Angiography is currently the modality of choice for the detection of CAD, however it has many side effects and is costly. Its complications and costs have prompted researchers to seek alternative methods for overcoming these deficiencies. The present invention addresses these and related needs.

### BRIEF SUMMARY

The present invention provides a system for determining the need for angiography in a patient. The system includes a pre-processing phase and a main phase. The main phase may include a data mining algorithm for the detection of Coronary Artery Disease. The pre-processing phase generates (i) LAD ratio, (ii) LCX ratio, and (iii) RCA ratio. These features are generated in a way to be correlated with blockage of Left Anterior Descending (LAD), Left Circumflex (LCX), or Right Coronary Artery (RCA). Higher values of any of these created features, indicates higher probability of having CAD. Each of these features is derived from the set of available features in the dataset.

The present invention also provides a computer-implemented method of data mining for determining the need for angiography in a patient. The method includes collecting first group of data features from a patient, wherein the data features are relevant for the detection of Coronary Artery Disease; comparing the first group of data features with a reference second group of data features that are relevant for the detection of Coronary Artery Disease; and generating report from the comparison, thereby determining the need for angiography in the patient. In the computer-implemented method of data mining, the groups of data features may be Z-AlizadehSani feature set.

### BRIEF DESCRIPTION OF THE DRAWINGS

FIG. 1, illustrates a Bayesian Network demonstrating relationship of the features of a patient, stenosis of the individual arteries and having CAD for him/her.

FIG. 2, describes schematically the used probabilistic method.

### DETAILED DESCRIPTION OF THE PRESENTLY PREFERRED EMBODIMENTS

In one aspect, the present invention provides for the classification of a patient into CAD or normal class, using drawn medical knowledge from the dataset mentioned herein, and therefore determining the need to perform angiography. CAD patients are in need of angiography; normal patients are not in need of angiography.

In one aspect, the invention provides a method for classifying a patient into CAD or normal class, where the method includes two steps: (1) a pre-processing phase; and (2) a main phase. The pre-processing phase comprises an algorithm for creation of three features derived from the mentioned dataset that are named LAD ratio, LCX ratio and RCA ratio with purpose of recognizing blockage in the three major coronary arteries. In order to create these features, firstly the most important features of the train dataset for recognizing the stenosis of the three arteries using PCA feature selection method are selected. The selection algorithm needs the real label of LAD, LCX and RCA stenosis in the train dataset which are provided to the algorithm. Afterwards, in the main phase, three classifiers are trained on these three sets of selected features. These classifiers predict the stenosis of LAD, LCX and RCA. The predictions of the three classifiers create three new features. The three created features therefore are added to the dataset in the main phase.

The main phase includes a data mining diagnosis method for predicting the stenosis in the major coronary arteries using the created features in the pre-processing phase. Classifiers named Cₓ, for x={LAD, LCX, and RCA} are used for diagnosing of stenosis. These are singled out based on a train dataset and the real label of the LAD, LCX, and RCA stenosis. In addition to the above three classifiers, a fourth classifier, C_{CAD} is created, which predicts having CAD. The predictions of these four classifiers are added to the train dataset.

In one aspect of the invention, a general classifier, namely ensemble CAD classifier, combining both the important selected features determined by the selection algorithm and the four added features, determines the need for angiography in a patient. The first four classifiers, i.e. C_{LAD}, C_{LCX}, C_{RCA} and C_{CAD}, i.e. are evaluated and their predictions are added in the test dataset where fifth classifier named ensemble CAD classifier which uses the results of other classifiers determines CAD employing the important and added features.

In another aspect of the invention, a method is provided for recognizing patients with or without CAD thus determining the need for angiography.

For accurate CAD diagnosis, it is sufficient to know the stenosis of the three arteries. Data mining diagnosis methods can be applied to predict the stenosis of the individual arteries. Indeed, a classifier can be used for stenosis diagnosis of the arteries. These classifiers are named Cₓ, for x={LAD, LCX, and RCA}. Depending on the accuracy of these classifiers, using the stenosis predictions for the arteries may increase or decrease the accuracy of the method.

In one preferred embodiment of the invention, the system uses the predictions of the classifiers of the arteries. These three classifiers are singled out based on a train dataset and the real label of the LAD, LCX, and RCA stenosis. In addition to these classifiers, a classifier is created on the train dataset for diagnosing CAD. These four classifiers that predict the stenosis of LAD, LCX, RCA and having CAD are added to the train dataset. A general classifier determines CAD using both important selected features of the dataset, which are determined using a feature selection algorithm, and the four added features. Subsequently, the first four classifiers are evaluated on the test dataset and the four features are added to this set. A fifth classifier, i.e. ensemble CAD classifier, determines CAD in the test dataset employing the important and added features. In this way, the important knowledge of the train data is used to train a classifier by using both the features of the dataset and the fact that the stenosis of each major artery bears having CAD for a patient.

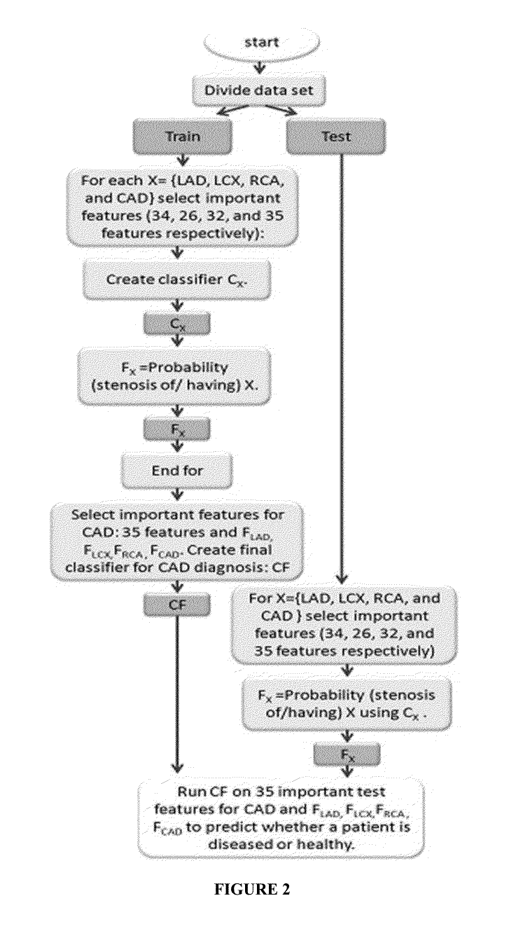

Instead of using the binary predictions of the four mentioned classifiers, their posterior probabilities may be used as the injected features. Using the probabilities instead of binary predictions yields better discrimination of patients, especially for ones whose arteries are partly stenotic. In one example, in order to divide the dataset to train and test sets, a 10-fold cross validation was used. In one preferred embodiment of the invention, the algorithm's pseudocode is described in FIG. 2, as an algorithm's flowchart.

In one embodiment, the present invention provides a feature set called Z-AlizadehSani with 50 features. This feature set is introduced which utilizes several effective features.

In one aspect, the present invention proposes a novel data mining algorithm for the detection of CAD. The algorithm outputs the probability of having CAD for its input and achieves an accuracy rate of 94.28% and sensitivity rate of 100% for the detection of CAD on 335 patients represented by Z-AlizadehSani feature set. To the best of our knowledge, such high rates of accuracy and sensitivity have not been attained elsewhere before.

The sensitivity rate of 100% makes the method a highly applicable one. In fact, a patient can be tested with this method, first. In case of negative result of the method, he/she can safely be sure that there is no need to angiography, based on the results of our dataset. Therefore, the side effects and costs of angiography can be avoided. Otherwise, angiography is recommended for him/her to determine the exact location and percent of the stenosis. The rate of 100% sensitivity means that if a sample was determined as healthy, he/she was healthy beyond reasonable doubt. Checking the false predictions of the algorithm, i.e. the healthy individuals determined as patients, showed that all of them had minimal CAD. So the error of this algorithm is mostly because of predicting some healthy people who have minimal CAD as CAD patients. Therefore this new algorithm reliably distinguishes patients with normal coronary arteries from those with CAD, obviating the need for angiography in the former group.

The Z-AlizadehSani feature set have been extracted for 335 patients. All features can be considered as indicators of CAD for a patient, according to medical literature. However, some of them have never been used in data mining based approaches for CAD diagnosis. The features are arranged in four groups: demographic, symptom and examination, ECG, and laboratory and echo features.

The description provides the features of Z-AlizadehSani feature set along with their valid ranges or the ranges of the features in the dataset, respectively. Each patient could be in two possible categories CAD or Normal. A patient is categorized as CAD, if his/her diameter narrowing is greater than or equal to 50%, and otherwise as Normal.

The discretization ranges provided in Braunwald heart book are also used to enrich the dataset with discretized versions of some existing features. These new features are indicated by index 2 and are depicted in Table 2. Experiments show that these features which have been drawn from medical knowledge could help the classification algorithms to better classify a patient into CAD or Normal class.

In one aspect, the present invention provides a novel data mining algorithm for the detection of CAD. The data mining algorithm may be a part of a system. The system may contain a pre-processing phase and a main phase. In the pre-processing phase, a novel algorithm is proposed for the creation of new features. In one embodiment, these three new features include: LAD ratio, LCX ratio, and RCA ratio. These features are specialized for recognizing whether three major coronary arteries, Left Anterior Descending (LAD), Left Circumflex (LCX), or Right Coronary Artery (RCA) are blocked, respectively. In general, higher values of any of these features (LAD ratio, LCX ratio, and RCA ratio) indicate higher probability of having CAD. Each of these features is derived from the set of available features in the dataset.

In one preferred embodiment of the invention, the data mining algorithm processes one dataset that contains 50 features which are indicators for CAD for a patient.

In another aspect of the invention, as illustrated in Table 1, the features of this invention are arranged in four groups: (i) demographic, (ii) symptom and examination, (iii) ECG, and (iv) laboratory and echo features. The method may use each of these categories of features. Table 1 presents one example of the features of the dataset along with their valid ranges or the ranges of the features in the dataset, respectively.

TABLE 1
Z-AlizadehSani feature set

| Feature Type | Feature Name | Range |
|---|---|---|
| Demographic | Age | 30-86 |
| | Weight | 48-120 |
| | Length | 150-210 |
| | Sex | Male, Female |
| | BMI (Body Mass Index Kg/m²) | 18-41 |
| | DM (Diabetes Mellitus) | Yes, No |
| | HTN (Hyper Tension) | Yes, No |
| | Current Smoker | Yes, No |
| | Ex-Smoker | Yes, No |
| | FH (Family History) | Yes, No |
| | Obesity | Yes if |
| | | MBI > 25, |
| | | No otherwise |
| | Thyroid Disease | Yes, No |
| | CHF (Congestive Heart Failure) | Yes, No |
| | DLP (Dyslipidemia) | Yes, No |
| Symptom and | BP (Blood Pressure: mmHg) | 90-190 |
| Examination | PR (Pulse Rate) (ppm) | 50-110 |
| | Weak peripheral pulse | Yes, No |
| | Systolic murmur | Yes, No |
| | Diastolic murmur | Yes, No |
| | Typical Chest Pain | Yes, No |
| | Dyspnea | Yes, No |
| | Function Class | 1, 2, 3, 4 |
| | Atypical | Yes, No |
| | Nonanginal CP | Yes, No |
| | Exertional CP (Exertional Chest | Yes, No |
| | Pain) | |
| | Low ThAng (low Threshold angina) | Yes, No |
| ECG | Rhythm | Sin, AF |
| | Q Wave | Yes, No |
| | ST Elevation | Yes, No |
| | ST Depression | Yes, No |
| | T inversion | Yes, No |
| | LVH (Left Ventricular Hypertrophy) | Yes, No |
| | Poor R Progression (Poor R Wave | Yes, No |
| | Progression) | |
| Laboratory | FBS (Fasting Blood Sugar) (mg/dl) | 62-400 |
| and Echo | Cr (creatine) (mg/dl) | 0.5-2.2 |
| | TG (Triglyceride) (mg/dl | 37-1050 |
| | LDL (Low density lipoprotein) | 18-232 |
| | (mg/dl) | |
| | HDL (High density lipoprotein) | 15-111 |
| | (mg/dl) | |
| | BUN (Blood Urea Nitrogen) (mg/dl) | 6-52 |
| | ESR (Erythrocyte Sedimentation | 1-90 |
| | rate) (mm/h) | |
| | HB (Hemoglobin) (g/dl) | 8.9-17.6 |
| | K (Potassium) (mEq/lit) | 3.0-6.6 |
| | Na (Sodium) (mEq/lit) | 128-156 |
| | WBC (White Blood Cell) (cells/ml) | 3700-18000 |
| | Lymph (Lymphocyte) (%) | 7-60 |
| | Neut (Neutrophil) (%) | 32-89 |
| | PLT (Platelet) (1000/ml) | 25-742 |
| | EF (Ejection Fraction) (%) | 15-60 |
| | Region with RWMA (Regional Wall | 0, 1, 2, |
| | Motion Abnormality) | 3, 4 |
| | VHD (Valvular Heart Disease) | Normal, Mild, |
| | | Moderate, |
| | | Severe |

The discretization ranges provided in Braunwald heart book are also used to enrich the dataset with discretized versions of some existing features. These new features are indicated by index 2 and are depicted in Table 2.

TABLE 2
Descritized features and their range of values

| Feature | Low | Normal | High |
|---|---|---|---|
| Cr2 | Cr < 0.7 | 0.7 ≦ Cr ≦ 1.5 | Cr > 1.5 |
| FBS2 | FBS < 70 | 70 ≦ FBS ≦ 105 | FBS > 105 |
| LDL2 | | LDL ≦ 130 | LDL > 130 |
| HDL2 | HDL < 35 | HDL ≧ 35 | — |
| BUN2 | BUN < 7 | 7 ≦ BUN ≦ 20 | BUN > 20 |
| ESR2 | | if male | if male |
| | | &ESR ≦ age/2 | & ESR > age/2 |
| | | or if female | or if female |
| | | &ESR ≦ age/2 + 5 | & ESR > age/2 + 5 |
| HB2 | if male & | if male & | if male & |
| | HB < 14 | 14 ≦ HB ≦ 17 | HB > 17 |
| | Or If | or if female | or if female |
| | female & | & 12.5 ≦ HB <= 15 | & HB > 15 |
| | HB < 12.5 | | |
| K2 | K < 3.8 | 3.8 ≦ K ≦ 5.6 | K > 5.6 |
| Na2 | Na < 136 | 136 ≦ Na ≦ 146 | Na > 146 |
| WBC2 | WBC < | 4000 ≦ WBC ≦ | WBC > 11000 |
| | 4000 | 11000 | |
| PLT2 | PLT < 150 | 150 ≦ PLT ≦ 450 | PLT > 450 |
| EF2 | EF ≦ 50 | EF > 50 | |
| Region | — | Region with | Region with |
| with | | RWMA = 0 | RWMA ≠ 0 |
| RWMA2 | | | |
| Age2* | | if male & | if male & |
| | | age ≦ 45 | age > 45 |
| | | or if female | or if female |
| | | & age ≦ 55 | & age > 55 |
| BP2 | BP < 90 | 90 ≦ BP ≦ 140 | BP > 140 |
| PulseRate2 | PulseRate < 60 | 60 ≦ PulseRate ≦ | PulseRate > 100 |
| | | 100 | |
| TG2 | | TG ≦ 200 | TG > 200 |
| Function | | 1 | 2, 3, 4 |
| Class2 | | | |

*Given thatwomenunder55 years and menunder45 yearsarelessaffected by CAD, the range of age is partitioned at these values.

In one preferred embodiment, the procedure 1 explains how to create LAD ratio in detail. Available features of the dataset are first discretized into binary variables. The system is designed according to an assumption about the descritized features: value 0.9 for a feature indicates higher probabilities of the record being in the CAD class, while value 0.1 indicates otherwise. LCX and RCA ratios are created with similar methods.

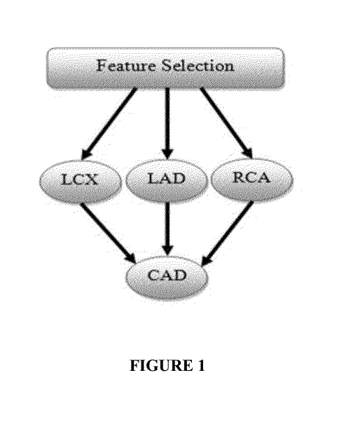

In one preferred embodiment, after applying the pre-processing phase of the algorithm, the main phase is performed as follows: according to the definition, if one of the left anterior descending coronary artery (LAD), left circumflex artery (LCX), or right coronary artery (RCA) is stenotic, the patient has CAD. The stenosis of these arteries of a patient is dependent on the other features of him/her. A Bayesian Network demonstrates the relationship of the features, stenosis of the individual arteries and having CAD for a patient (FIG. 1).

The data mining algorithm processes one or more datasets that contain a number of features that are indicators for CAD in a patient. The number of features that are indicators for CAD in a patient is preferably between 20 and 100 features, more preferably between 40 and 80 features, most preferably 50 features. Features that may be used in the practice of the present invention include but are not limited to those features shown in Table 1. The exemplary features shown in Table 1 are in this embodiment arranged in four groups: (1) demographic, (2) symptom and examination, (3) ECG, and (4) laboratory and echo features. These features and/or groups are non-limiting, and in the practice of the present invention it would be possible to use additional features familiar to those skilled in the art, and to also arrange the features in different groups. In one example, Table 1 presents the features of the inventor dataset along with their valid ranges or the ranges of the features in the dataset, respectively.

Some aspects of the present invention are also described in the article by Alizadehsani et al., A data mining approach for diagnosis of coronary artery disease, Comput Methods Programs Biomed. 2013 July; 111(1):52-61. doi: 10.1016/j.cmpb.2013.03.004. Epub 2013 Mar. 25, which is also incorporated herein by reference. It should be noted that the current invention is completely different and better than the features provided in the aforementioned article.

After applying the pre-processing phase of the algorithm, the main phase is performed as follows: according to the definition, if one of the left anterior descending coronary artery (LAD), left circumflex artery (LCX), or right coronary artery (RCA) is stenotic, the patient has CAD. The stenosis of these arteries of a patient is dependent on the other features of him/her. A Bayesian Network demonstrates the relationship of the features, stenosis of the individual arteries and having CAD for a patient (FIG. 1).

In one embodiment, classifier are used for stenosis diagnosis of the arteries. These classifiers are named Cₓ, for x={LAD, LCX, and RCA}. The system uses the predictions of the classifiers of the arteries. These three classifiers are singled out based on a train dataset and the real label of the LAD, LCX, and RCA stenosis. In addition to these classifiers, a classifier is created on the train dataset for diagnosing CAD. These four classifiers that predict the stenosis of LAD, LCX, RCA and having CAD are added to the train dataset. A general classifier determines CAD using both important selected features of the dataset, which are determined using a feature selection algorithm, and the four added features. Subsequently, the first four classifiers are evaluated on the test dataset and the four features are added to this set. A fifth classifier, named C_{P}, determines CAD in the test dataset employing the important and added features.

In one embodiment of the invention, instead of using the binary predictions of the four mentioned classifiers, their posterior probabilities are used as the injected features. In one example, in order to divide the dataset to train and test sets, 10-fold cross validation was used. The algorithm's pseudocode is therefore as follows (see also Figure for algorithm's flowchart):
1. Use 10-fold cross validation to divide the dataset into two parts: 0.9 for train data and 0.1 for test data.
2. On the train data: select important features for the classification of x={LAD, LCX, RCA, and CAD} (34, 26, 32, and 35 features, respectively) and create classifier Cₓ for X. Define Fₓ=Probability (stenosis of/having) x running classifier C on train features.
3. Using the selected features for CAD and F_{LAD}, F_{LCX}, F_{RCA}, and F_{CAD} as the new features, create a classifier for predicting whether a patient has CAD or is normal. Name this classifier as Cp.
4. For each test data:Run C_{P} classifier on the selected features and F_{LAD}, F_{LCX}, F_{RCA}, and F_{CAD} (which are derived from Cₓ for the test data) to predict whether the sample has CAD or is normal.
5. On the train data: select important features for the classification of x={LAD, LCX, RCA, and CAD} (34, 26, 32, and 35 features, respectively) and create classifier Cₓ for X. Define Fₓ=Probability (stenosis of/having) x running classifier C on train features.
6. Using the selected features for CAD and F_{LAD}, F_{LCX}, F_{RCA}, and F_{CAD} as the new features, create a classifier for predicting whether a patient has CAD or is normal. Name this classifier as C_{P}.
7. For each test data:run C_{P} classifier on the selected features and F_{LAD}, F_{LCX}, and F_{CAD} (which are derived from Cₓ for the test data) to predict whether the sample has CAD or is normal.

The following displays procedure 1, creating features in the pre-process phase: On train data:

1. For any feature f do

convert f to a binomial feature using the following steps:
a. If f is numerical, discretize it by breaking its domain into intervals.
b. If f is binomial, feature values are considered as 0.1 and 0.9. The values that have positive effect on Cad are considered as 0.9 and the others considered as 0.1.
c. If f is polynomial, change it to binomial by mapping the values having direct relationship to CAD, to 0.9 and others to 0.1:

2. For all fεfeatures calculate the following fraction on training data:

w(f)=P(LAD=1|f=0.9)

3. Where LAD=1 means that LAD is clogged. Choose K features that have the highest W value. Name as f₁, f₂, . . . , f_{K}.
(K is set to 20 in the experiments.)

4. Compute

LAD ratio=sigmoid(W,F), where W=(w1, . . . wk),F=(f1, . . . fk)

In one preferred embodiment, the data mining algorithm also outputs the probability of having CAD for its input and achieves an accuracy rate of up to 94.28% and sensitivity rate of 100% for the detection of CAD using Z-AlizadehSani feature set for 335 patients. 100% sensitivity shows all CAD patients are recognized and if a person is recognized as normal, he/she is definitely normal. Therefore the algorithm can be used to determine need to angiography: If a sample is recognized as normal, there is no need to angiography; otherwise he/she should use angiography for determining the place and amount of stenosis. With respect to high cost and side effects of angiography, removing the need to angiography for most of normal patients is an invaluable work in medicine. In addition to the Z-AlizadehSani feature set, other features obvious to one skilled in the art may be used to practice the present invention.

The features described herein are specialized for recognizing whether three major coronary arteries, Left Anterior Descending (LAD), Left Circumflex (LCX) or Right Coronary Artery (RCA) is blocked, respectively. Higher values of any of these created features, indicates higher probability of having CAD. Each of these features is derived from the set of available features in the dataset.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of chemistry, biochemistry, molecular biology, and bioengineering, are within the scope of this invention. All publications, patents, and patent applications cited herein are incorporated by reference in their entirety for all purposes.

1- A system for determining the need for angiography in a patient, comprising a pre-processing phase and a main phase,
wherein said pre-processing phase comprises an algorithm for generation of three features; a (i) LAD (Left Anterior Descending) ratio, (ii) LCX (Left Circumflex) ratio, and (iii) RCA (Right Coronary Artery) ratio, and wherein said main phase comprises a data mining algorithm for detection of Coronary Artery Disease; wherein said system distinguishes patients with normal coronary arteries (NCA) from those with CAD (Coronary Artery Disease), obviating a need for angiography in said patients with NCA, wherein said system comprises a sensitivity rate of 100%.

2- The system of claim 1, wherein said three features are selected based on PCA feature selection method and wherein in said main phase, three classifiers are trained on said three sets of selected features; wherein said classifiers predict stenosis of LAD, LCX and RCA; wherein predictions of said three classifiers create three new features and then are added to the dataset in the main phase.

3- The system of claim 2, wherein the LAD ratio, LCX ratio and RCA ratio are three features derived from Z-AlizadehSani feature set wherein their corresponding values correlate with blockage of LAD, LCX and RAD.

4- The system of claim 3, wherein Z-AlizadehSani data features are selected from a group consisting of: age, weight, sex, body mass index, diabetes mellitus, hyper tension, current smoker, ex-smoker, family history, obesity, chronic renal failure, cerebrovascular accident, thyroid disease, congestive heart failure, dyslipidemia, blood pressure, pulse rate, weak peripheral pulse, systolic murmur, diastolic murmur, typical chest pain, dyspnea, function class, atypical chest pain, nonanginal chest pain, exertional chest pain, low threshold angina, rhythm, Q wave, ST elevation, ST depression, T inversion, left ventricular hypertrophy, poor R wave progression, fasting blood sugar, creatine, triglyceride, low density lipoprotein, high density lipoprotein, blood urea nitrogen, erythrocyte sedimentation rate, hemoglobin, potassium, sodium, white blood cell count, lymphocyte count, neutrophil, platelets, ejection fraction, region wall motion abnormality, and valvular heart disease.

5- System for determining angiography procedure for a patient using general ensemble CAD classifier combining both selected features determined by a selection algorithm from a Z-AlizadehSani dataset and four added features from four classifiers C_{LAD}, C_{LCX}, C_{RCA} and C_{CAD}.

6- A computer-implemented method of data mining for determining the need for angiography in a patient, comprising the steps of:
Collecting first group of data features from a patient, wherein said data features are relevant for detection of Coronary Artery Disease;
comparing said first group of data features with a reference second group of data features that are relevant for detection of Coronary Artery Disease;
and generating a report from said comparison, thereby determining a need for angiography in a patient,
wherein said data features indicate whether any one of three major coronary arteries, Left Anterior Descending (LAD), Left Circumflex (LCX), or Right Coronary Artery (RCA) is blocked, respectively, and wherein relatively higher values of said data features indicate higher probability of having Coronary Artery Disease.

7- The method of claim 6, wherein said data features are derived from a Z-AlizadehSani feature set.

8- The method of claim 7, wherein said data features are selected from a group consisting of: age, weight, sex, body mass index, diabetes mellitus, hyper tension, current smoker, ex-smoker, family history, obesity, chronic renal failure, cerebrovascular accident, thyroid disease, congestive heart failure, dyslipidemia, blood pressure, pulse rate, weak peripheral pulse, systolic murmur, diastolic murmur, typical chest pain, dyspnea, function class, atypical chest pain, nonanginal chest pain, exertional chest pain, low threshold angina, rhythm, Q wave, ST elevation, ST depression, T inversion, left ventricular hypertrophy, poor R wave progression, fasting blood sugar, creatine, triglyceride, low density lipoprotein, high density lipoprotein, blood urea nitrogen, erythrocyte sedimentation rate, hemoglobin, potassium, sodium, white blood cell count, lymphocyte count, neutrophil, platelets, ejection fraction, region wall motion abnormality, and valvular heart disease.
